(19) United States
(12) Patent Application Publication
(10) Pub. No.: US 2019/0346423 A1
(43) Pub. Date: Nov. 14, 2019

(54) METHODS FOR EVALUATING MONOCLONALITY

(71) Applicant: LONZA LTD, Visp (CH)

(72) Inventors: Hilary K. Metcalfe, Radnage (GB); Adekunle O. Onadipe, Ballwin, MO (US); Andrew J. Racher, Brightwalton Newbury (GB); Alison Porter, Charvil (GB)

(21) Appl. No.: 16/478,541

(22) PCT Filed: Jan. 17, 2018
(86) PCT No.: PCT/US18/13941

§ 371 Date: Jul. 17, 2019

Related U.S. Application Data
(60) Provisional application No. 62/447,724, filed on Jan. 18, 2017.
(60) Provisional application No. 62/505,293, filed on May 12, 2017.

(51) Int. Cl. G01N 33/487; G01N 15/14; G01N 1/14

(52) U.S. Cl. CPC G01N 33/48735; G01N 15/1463; G01N 2001/149; G01N 2015/149; G01N 2015/1488; G01N 1/14

(57) ABSTRACT

Disclosed are methods for evaluating a value of probability of monoclonality of populations of cells.

### RELATED APPLICATIONS

This application claims priority to U.S. Ser. No. 62/447,724, filed Jan. 18, 2017, and U.S. Ser. No. 62/505,293, filed May 12, 2017, the entire contents of which are incorporated herein by reference in their entirety.

### FIELD OF THE INVENTION

The present disclosure relates to methods of evaluating the probability of monoclonality in the growth of aliquots identified as containing a single cell. The present disclosure also relates to the evaluation of the reliability of methods of producing monoclonal cell lines to produce therapeutic polypeptides.

### BACKGROUND

Ensuring clonality of a cell line is fundamental to qualitative and quantitative cell culture science and economics of manufacture. A cell line that is not clonal may not be consistent and reliable for manufacturing use. It is also a regulatory expectation that a cloning procedure has been used in the preparation or derivation of the production cell line. Recently, there has been increased scrutiny of the methods used to achieve monoclonality, with concerns expressed over certain approaches taken.

Limiting dilution is a commonly used cell cloning method which relies on statistical distribution (Puck & Marcus, 1955). A limitation of this technique is that while the seeding of the cells follows a Poisson distribution, the number of colonies observed does not (Underwood & Bean, 1988; Coller & Coller, 1986). Therefore, to achieve an acceptable level of probability of monoclonality, multiple rounds of limiting dilution cloning are typically required. As the creation of a clonal cell line is often a critical path activity during therapeutic product development, alternative methods have been developed that enable faster derivation of clonal cell lines using a single round of cloning. These methods include the “spotting” technique, fluorescence activated cell sorting, and cloning rings. The capillary-aided cell cloning technique was developed as a variation of the “spotting” technique described by Clarke & Spier, 1980.

Florescence activated cell sorting (FACS) has been used to quickly isolate single cells, with a high probability of monoclonality achieved in a single cloning round instead of the multiple rounds required with the limiting dilution method. Typically, there has been reliance upon the vendor's data and recommendations to support FACS set-up for single-cell sorting.

The capillary-aided cell cloning (CACC) technique involves the use of a capillary tube to dispense droplets of a dilute cell suspension into multi-well plates. Typically two scientists independently visually inspect the droplets for the number of cells contained therein. Colonies found growing in the wells where both scientists independently reported the observation of a single cell during the cloning are considered to be monoclonal.

The use of the capillary aided cell cloning technique offers a number of advantages, but regardless of cell cloning method, there exists a need to assess the reliability of the production of clonal cell lines; in other words, to evaluate the probability that a cell line identified to be monoclonal is in fact monoclonal.

### SUMMARY OF THE INVENTION

The present disclosure is based, in part, on the discovery that it is possible to evaluate a value of the probability of monoclonality of the growth of aliquots identified as containing a single cell amongst a plurality of aliquots distributed from a cell population provided in the process of cell line production. Methods disclosed herein provide for the evaluation of the reliability of methods of producing monoclonal cell lines to produce therapeutic polypeptides, and allow increased confidence the monoclonality of a broad variety of methods of producing monoclonal cell lines. Without wishing to be bound by theory, it is believed that calculations of data values for the frequencies at which aliquots were identified as having zero, one, or more cells, and whether the aliquots showed or did not show subsequent growth, can be applied to a probability equation, generating a value for the probability that growth from an aliquot identified as containing one cell is monoclonal growth. Accordingly, disclosed herein are methods for evaluating a value for probability of monoclonality. These methods include providing a solution comprising a population of cells, forming a plurality of aliquots of the solution, identifying aliquots having zero, one, or more cells, and providing, for aliquots identified as having one cell, a value for the probability that subsequent growth was monoclonal. Thus provided herein are also exemplary cell lines, methods of forming a plurality of aliquots, methods of identifying the numbers of cells in aliquots, and methods for providing a value for the probability of monoclonality. Methods disclosed herein can be applied to improve any of a variety of methods for achieving monoclonality, including methods, such as CACC which even without the use of the methods described here give acceptable and even very good results. The methods described herein can be used with methods for achieving monoclonality that rely on direct human inspection for the presence or absence of cells or machine-based, e.g., computer-based image analysis for the detection of the presence or absence of cells. Methods described herein can improve reliability of the performance of machine-based scoring.

Accordingly, in one aspect, the invention features a method of evaluating a value for probability of monoclonality, comprising: providing a solution comprising a population of cells; forming a plurality of aliquots of the solution; identifying aliquots having one cell; and providing, for aliquots identified as having one cell, a value for the probability that subsequent growth was monoclonal, thereby evaluating a value for probability of monoclonality.

In an embodiment, forming a plurality of aliquots of the solution is accomplished using a printing device, by pipetting, using a capillary device (e.g., as in CACC), or using fluorescence-activated cell sorting (FACS) or flow cytometry.

In an embodiment, forming a plurality of aliquots of the solution is accomplished using a capillary device (e.g., as in CACC).

In an embodiment, forming a plurality of aliquots of the solution is accomplished using FACS or flow cytometry.

In an embodiment, identifying aliquots having one cell is accomplished using FACS or flow cytometry.

In an embodiment, forming a plurality of aliquots of the solution and identifying aliquots having one cell is accomplished using FACS or flow cytometry.

In an embodiment, an observer, e.g., a human observer or a machine observer:

a) identifies the number of cells in a plurality of aliquots, including e.g., the number of aliquots having 0, 1, or more than one cells;

b) identifies aliquots having one cell and identifies whether an aliquot shows subsequent growth;

c) memorializes a value for b) or c).

In an embodiment an observer, e.g., a human observer or a machine observer performs a).

In an embodiment an observer, e.g., a human observer or a machine observer performs a) and b).

In an embodiment an observer, e.g., a human observer or a machine observer performs a), b) and c).

In an embodiment, a second observer, e.g., a second human observer or a second machine observer (or a second use of the machine observer) performs one or more of a), b), and c), e.g., a), a) and b), or a), b), and c).

In an embodiment, the observer and a second observer, e.g., a second human observer or a second machine observer (or a second use of the machine observer), both performs one or more of a), b), and c), e.g., a), a) and b), or a), b), and c).

In an embodiment, a plurality of, e.g., two, observers, e.g., a plurality of, e.g., two human observers, a plurality of, e.g., two, machine observers (or a second use of the machine observer), or a human observer and a machine observer, identifies aliquots having one cell and identify whether an aliquot shows subsequent growth.

In an embodiment, two observers identify aliquots having one cell and identify whether an aliquot shows subsequent growth.

In an embodiment, two observers identify whether an aliquot has zero, one, or more cells, and identify whether an aliquot shows subsequent growth.

In an embodiment, the value assigned to an aliquot by an observer is memorialized.

In an embodiment, the value assigned to an aliquot by a second observer is memorialized.

In an embodiment, the value assigned to an aliquot by an observer and a second observer is memorialized if it meets a preselected criterion. In an embodiment, the criterion is that the value assigned by the first observer and value assigned by the second observer are identical, e.g., they both score an aliquot as having a single cell. In an embodiment the criterion is that the value assigned by the first observer and value assigned by the second observer are not identical, e.g., if one scores the cell as having one cell and the other scores the aliquot as having a value other than one cell.

In an embodiment, providing, for aliquots identified as having one cell, a value for the probability that subsequent growth was monoclonal, comprises calculating data values for the frequencies at which aliquots were identified as having zero, one, or more cells, and whether the aliquots showed or did not show subsequent growth; and using a probability equation and the data values to evaluate the probability that the subsequent growth of an aliquot identified as having one cell is monoclonal.

In an embodiment, providing, for aliquots identified as having one cell, a value for the probability that subsequent growth was monoclonal, comprises calculating data values for the frequencies at which aliquots were identified as having zero, one, or more cells, and whether the aliquots showed or did not show subsequent growth, the data values comprising the data values listed in Table 6.

In an embodiment, providing, for aliquots identified as having one cell, a value for the probability that subsequent growth was monoclonal, comprises calculating data values for the frequencies at which aliquots were identified as having zero, one, or more cells, and whether the aliquots showed or did not show subsequent growth, the data values comprising: n₀₁, the number of aliquots two observers identified as containing zero cells that did not show subsequent growth; n₀₂, the number of aliquots one observer identified as containing zero cells and one observer identified as containing one cell that did not show subsequent growth; n₀₃, the number of aliquots two observers identified as containing one cell that did not show subsequent growth; n₀₄, the number of aliquots one observer identified as containing zero cells and one observer identified as containing more than one cell that did not show subsequent growth; n₀₅, the number of aliquots one observer identified as containing one cell and one observer identified as containing more than one cell that did not show subsequent growth; n₀₆, the number of aliquots two observers identified as containing more than one cell that did not show subsequent growth; n₁₁, the number of aliquots two observers identified as containing zero cells that showed subsequent growth; n₁₂, the number of aliquots one observer identified as containing zero cells and one observer identified as containing one cell that showed subsequent growth; n₁₃, the number of aliquots two observers identified as containing one cell that showed subsequent growth; n₁₄, the number of aliquots one observer identified as containing zero cells and one observer identified as containing more than one cell that showed subsequent growth; n₁₅, the number of aliquots one observer identified as containing one cell and one observer identified as containing more than one cell that showed subsequent growth; and n₁₆, the number of aliquots two observers identified as containing more than one cell that showed subsequent growth.

In an embodiment, providing, for aliquots identified as having one cell, a value for the probability that subsequent growth was monoclonal, comprises fitting/applying the data values to a probability equation comprising unknowns consisting of the parameters listed in Table 7 to evaluate the probability that the subsequent growth of an aliquot identified as having one cell is monoclonal.

In an embodiment, providing, for aliquots identified as having one cell, a value for the probability that subsequent growth was monoclonal, comprises fitting/applying the data values to a probability equation comprising unknowns consisting of: q₀₀, the probability of an observer identifying an aliquot as containing zero cells when the aliquot actually contains zero cells; q₁₀, the probability of an observer identifying an aliquot as containing zero cells when the aliquot actually contains one cell; q₀₁, the probability of an observer identifying an aliquot as containing one cell when the aliquot actually contains zero cells; q₁₁, the probability of an observer identifying an aliquot as containing one cell when the aliquot actually contains one cell; q₂₁, the probability of an observer identifying an aliquot as containing one cell when the aliquot actually contains more than one cell; μ, the mean number of cells in an aliquot; and p, the probability a cell will grow into observable growth, to evaluate the probability that the subsequent growth of an aliquot identified as having one cell is monoclonal.

In an embodiment, providing, for aliquots identified as having one cell, a value for the probability that subsequent growth was monoclonal, comprises fitting/applying the data values to a probability equation consisting of

$$P = \frac{2 {q}_{11}^{2} + 2 ( 1 - p ) {q}_{21}^{2} μ}{2 {q}_{11}^{2} + ( 2 - p ) {q}_{21}^{2} μ}$$

to evaluate the probability that the subsequent growth of an aliquot identified as having one cell is monoclonal.

In an embodiment, providing, for aliquots identified as having one cell, a value for the probability that subsequent growth was monoclonal, comprises fitting/applying the data values to a probability equation comprising unknowns consisting of the parameters listed in Table 7 to evaluate the probability that the subsequent growth of an aliquot identified as having one cell is monoclonal, wherein more than one (e.g. two, three, four, five, six, or more) sets of starting values for the unknowns are used to apply the data values to the probability equation.

In an embodiment, providing, for aliquots identified as having one cell, a value for the probability that subsequent growth was monoclonal, further comprises assessing the evaluation of the probability using one or more statistical analyses, e.g. maximum likelihood, minimum sum of squares, minimum chi-squared, or log-likelihood ratio, wherein a higher maximum likelihood, lower minimum sum of squares, lower minimum chi-squared, and lower log-likelihood ratio indicate a more reliable evaluation of the probability.

In an embodiment, the invention features a method of evaluating the reliability of a single cell cloning technique, comprising: a) providing a solution comprising a population of cells; b) performing a first estimate of the value of the probability of monoclonality of the single cell cloning technique, comprising: i) forming a plurality of aliquots of the solution; ii) identifying aliquots having one cell; and iii) providing, for aliquots identified as having one cell, a value of the probability that subsequent growth was monoclonal, c) practicing the single cell cloning technique for an interval, d) performing a second estimate of the value of the probability of monoclonality of the single cell cloning technique, comprising: i) forming a plurality of aliquots of the solution; ii) identifying aliquots having one cell; and iii) providing, for aliquots identified as having one cell, a value of the probability that subsequent growth was monoclonal; and e) comparing the first and second estimates of the value of the probability of monoclonality of the single cell cloning technique, thereby evaluating the reliability of a single cell cloning technique. In another embodiment, the method further comprises adjusting the single cell cloning technique to improve the value of the probability of monoclonality.

In an embodiment, the b) ii) and d) ii) comprise identifying aliquots having zero, one, or more cells.

In an embodiment, b) ii) and d) ii) comprise identifying aliquots having zero, one, or more cells using fluorescence microscopy.

In an embodiment, b) ii) and d) ii) comprise a plurality of observers identifying aliquots having zero, one, or more cells using fluorescence microscopy.

In an embodiment, b) ii) and d) ii) comprise two observers identifying aliquots having zero, one, or more cells using fluorescence microscopy.

In an embodiment, the observers identify an aliquot having zero, one, or more cells based on examining the same fluorescence micrograph of the aliquot.

In an embodiment, the observers identify an aliquot having zero, one, or more cells based on examining different fluorescence micrographs of the aliquot, e.g., a distinct fluorescence micrograph for each observer.

In an embodiment, the observers further identify whether an aliquot shows subsequent growth.

In an embodiment, b) iii) and d) iii) comprise:

a) calculating data values for the frequencies at which aliquots were identified as having zero, one, or more cells, and whether the aliquots showed or did not show subsequent growth; and

b) using a probability equation and the data values to evaluate the probability that the subsequent growth of an aliquot identified as having one cell is monoclonal.

In an embodiment, the single cell cloning technique is chosen from CACC, FACS, or spotting. In an embodiment, the single cell cloning technique is CACC. In an embodiment, the single cell cloning technique is FACS. In an embodiment, the single cell cloning technique is spotting.

In an embodiment, the interval comprises a number of aliquots formed without evaluating a value of the probability of monoclonality. In an embodiment, the number of aliquots is at least 1, 10, 50, 100, 200, 500, 1000, 1500, 2000, 2500, 3000, or more.

In an embodiment, the interval comprises a number of multi-well plates, e.g., 96-well plates, filled with aliquots without evaluating a value of the probability of monoclonality. In an embodiment, the number of multi-well plates, e.g., 96 well plates, is at least 1, 5, 10, 15, 20, 25, 30, or more.

In an embodiment, the steps of the method take the form of: a), b), [c), d), e)]ₙ, wherein [c), d), e)] is repeated n times, and wherein n is greater than or equal to 1. In an embodiment, n is greater than or equal to 2, 3, 4, 5, 6, 7, 8, 9, or 10.

In another aspect, the invention features, a method of evaluating the reliability of a single cell cloning technique, comprising:

a) providing a solution comprising a population of cells;

b) using a first method, e.g., CACC, or FACS, to form a plurality of aliquots of the solution, the plurality of aliquots comprising
i) a type 1 aliquot (or a sub-plurality of type 1 aliquots), having a first (or type 1) characteristic;
ii) a type 2 aliquot (or a sub-plurality of type 2 aliquots), having a second (or type 2) characteristic;

c) using the first observer, e.g., a machine observer, to evaluate the number of cells in the type 1 aliquot (or in aliquots of the sub-plurality of type 1 aliquots) and the number of cells in the type 2 aliquot (or in aliquots of the sub-plurality of type 2 aliquots);

d) providing, for aliquots identified in c) as having one cell, a value of the probability that subsequent growth was monoclonal,

e) using a second observer, e.g., a human observer, to evaluate the number of cells in the type 1 aliquot (or in aliquots of the sub-plurality of type 1 aliquots) and the number of cells in the type 2 aliquot (or in aliquots of the sub-plurality of type 2 aliquots);

f) providing, for aliquots identified in e) as having one cell, a value of the probability that subsequent growth was monoclonal; and

g) evaluating the value in d), f) or both,

thereby evaluating the reliability of a single cell cloning technique.

In an embodiment, g) comprises comparing the value from d), f) or both with a reference or threshold value, e.g., a threshold value of the probability of monoclonality.

In an embodiment, g) comprises comparing the value from d) with the value from f).

In an embodiment comparing comprises determining if the value from d), f) or both, nave a predetermined relationship with a reference or threshold value, e.g., determining if the value is less than, the same as, or exceed the reference or threshold value.

In an embodiment the first observer comprises a machine observer.

In an embodiment the second observer comprises a human observer.

In an embodiment the first observer comprises a machine observer and the second observer comprises a human observer.

In an embodiment, the method comprises providing an image of a plurality of aliquots evaluated by the first observer and the second observer reads the image to evaluate the plurality of aliquots.

In an embodiment the first or type 1 characteristic comprises aliquots formed in a first time period and the second or type 2 characteristic comprises aliquots formed in a second time period.

In an embodiment the type 1 aliquot (or a sub-plurality of type 1 aliquots), was formed prior to the type 2 aliquot (or a sub-plurality of type 2 aliquots).

In an embodiment the type 1 aliquot (or a sub-plurality of type 1 aliquots), was evaluated for clonality prior to the type 2 aliquot (or a sub-plurality of type 2 aliquots).

In an embodiment the first or type 1 characteristic comprises aliquots formed in a first region of a substrate and the second or type 2 characteristic comprises aliquots formed in second region of a substrate.

In an embodiment the first region of a substrate comprises an aliquot adjacent to a border of the substrate and the second or type 2 characteristic comprises an aliquot not adjacent to a border of the substrate.

In an embodiment, b) comprises forming iii) a type 3 aliquot (or a sub-plurality of type 3 aliquots), having a third (or type 3) characteristic;

In an embodiment, a type 3 aliquot was formed after formation of a type 1 aliquot but prior to a type 2 aliquot.

In an embodiment, the method allows evaluation of the consistency of the first observer evaluations over a plurality of evaluations.

In an embodiment, c) and/or e) comprise identifying aliquots having zero, one, or more cells.

In an embodiment, c) and/or e) comprise identifying aliquots having zero, one, or more cells using fluorescence microscopy.

In an embodiment, c) and/or e) comprise a plurality of observers identifying aliquots having zero, one, or more cells using fluorescence microscopy.

In an embodiment, c) and/or e) comprise two observers identifying aliquots having zero, one, or more cells using fluorescence microscopy.

In an embodiment, c) and/or e) comprise observers identifying an aliquot having zero, one, or more cells based on examining the same fluorescence micrograph of the aliquot.

In an embodiment, c) and/or e) comprise identifying an aliquot having zero, one, or more cells based on examining different fluorescence micrographs of the aliquot, e.g., a distinct fluorescence micrograph for each observer.

In an embodiment, c) and/or e) comprise observers further identifying whether an aliquot shows subsequent growth.

In an embodiment, c) and/or e) comprise:

a) calculating data values for the frequencies at which aliquots were identified as having zero, one, or more cells, and whether the aliquots showed or did not show subsequent growth; and

b) using a probability equation and the data values to evaluate the probability that the subsequent growth of an aliquot identified as having one cell is monoclonal.

In an embodiment, the first method comprises a single cell cloning technique is chosen from CACC, FACS, or spotting. In an embodiment, the single cell cloning technique is CACC. In an embodiment, the single cell cloning technique is FACS. In an embodiment, the single cell cloning technique is spotting

In an embodiment, the type 3 aliquots are formed without evaluating a value of the probability of monoclonality. In an embodiment, the number of aliquots is at least 1, 10, 50, 100, 200, 500, 1000, 1500, 2000, 2500, 3000, or more.

In an embodiment, a number of multi-well plates, e.g., 96-well plates, are filled with aliquots without evaluating a value of the probability of monoclonality. In an embodiment, the number of multi-well plates, e.g., 96 well plates, is at least 1, 5, 10, 15, 20, 25, 30, or more.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc, are presented merely for ease of reading and are not limiting. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIG. 1 shows a graph of experimentally observed data compared with data predicted by the statistical model for wells showing cell growth after the cloning of a mixed culture of two GS-NS0 cell lines using the Capillary-Aided Cell Cloning technique. The horizontal axis represents paired observations of the number of cells reported by two scientists.

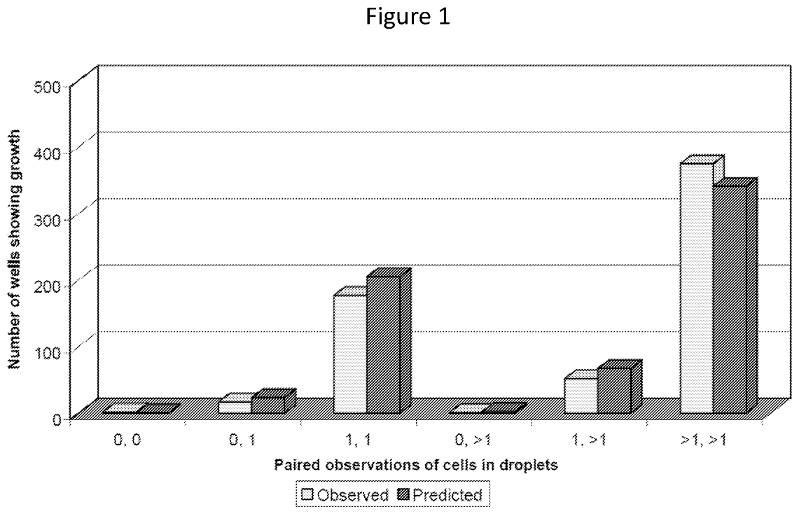

FIG. 2 shows a graph of experimentally observed data compared with data predicted by the statistical model for wells showing no cell growth after the cloning of a mixed culture of two GS-NS0 cell lines using the Capillary-Aided Cell Cloning technique. The horizontal axis represents paired observations of the number of cells reported by two scientists.

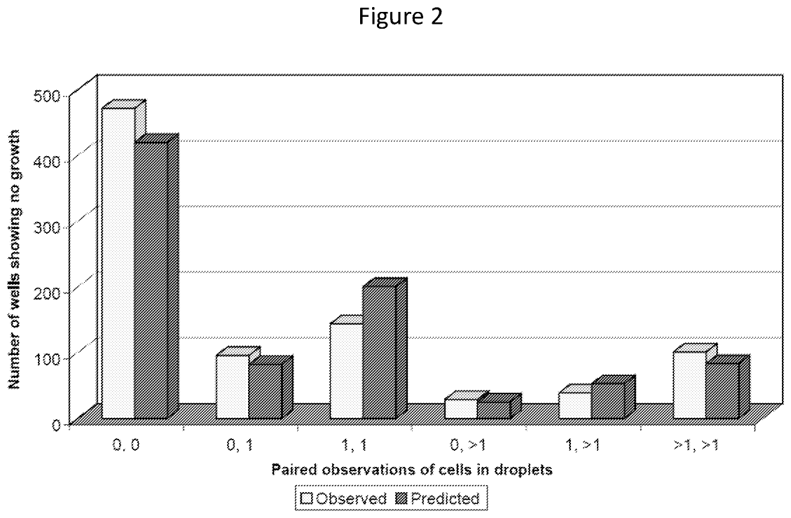

FIG. 3 shows FACS data depicting an exemplary gating strategy that excludes non-viable cells, debris, and doublet and higher order aggregates of cells.

FIG. 4 shows a schematic of positioning of a cell within the flow of solution being sorted or not sorted into droplets by the FACS instrument.

FIG. 5 shows a diagram depicting checking a well for the presence of 0, 1, or 2+ cells using fluorescence microscopy.

FIG. 6 shows a graph of exemplary past FACS instrument performance used to predict the probability of monoclonality of sample data.

FIG. 7 shows a graph of beta distributions of prior and posterior data of P(X=0).

FIG. 8 shows a graph of beta distributions of prior and posterior data of P(X=1).

FIG. 9 shows a graph of the probability of monoclonality per session on the FACS instrument as estimated as the mode of the posterior distribution.

FIG. 10 shows an image of a ˜1 μl droplet of cell suspension in a well, deposited by capillary action from a pipette tip.

FIGS. 11A-11C show images of droplets with 0 (FIG. 11A), 1, (FIG. 11B), or 2 (FIG. 11C) cells per droplet.

FIGS. 12A-12D show images of droplets that would be excluded from analysis. The droplet in FIG. 12A contains an air bubble, the droplet in FIG. 12B cannot be completely visualized in a single field of view, the droplet in FIG. 12C has touched the edge of the well (e.g., the boundary of the droplet is not clear), and the droplet in FIG. 12D contains debris.

### DETAILED DESCRIPTION OF THE INVENTION

### Definitions

The articles “a” and “an” are used herein to refer to one or to more than one (i.e., to at least one) of the grammatical object of the article. By way of example, “a cell” can mean one cell or more than one cell.

As used herein, the term “monoclonality” refers to a quality of a group of cells, wherein the quality is that the group of cells originated from exactly one parent cell. For example, a monoclonal cell line is a cell line that originated from exactly one cell.

As used herein, the term “value for probability of monoclonality” refers to an estimate of the likelihood that a group of cells identified as monoclonal is actually monoclonal.

As used herein, the term “aliquot” refers to a volume of a solution. In an embodiment, a plurality of aliquots are formed, examined or analyzed, and each aliquot of the plurality satisfies a condition with regard to volume, e.g., each aliquot of the plurality has: a minimal volume, e.g., a preset minimal value; falls within a range between a minimal and a maximal value, e.g., a preset minimal and/or maximal value; approximately equal values, e.g., a preset value; or the same volume, e.g., a preset value. In an embodiment the volume of an aliquot is constrained to volumes which meet a functional limitation. By way of example, each aliquot of a plurality of aliquots must fill a predetermined field of view for a human or machine observer, e.g., each must fill the entire field of view, e.g., the field of view formed using a microscope. When a larger amount of a liquid is divided into a plurality of aliquots, the plurality may be equal to the entire larger amount, or to less than the entire larger amount.

As used herein, the term “plurality of aliquots” refers to more than one (e.g., two or more) aliquots.

As used herein, the term “observer” refers to an entity capable of making an observation regarding the presence or absence of cells in an aliquot. The entity may be a human of sufficient skill. Typically a human observer makes a conclusion of cell number or growth baed on direct visual inspection of the aliquot, e.g, through a magnifying device. The entity may be a machine, e.g., a computerized device for forming and analyzing images, or other suitable automated device, e.g., a computerized microscope camera or the detector of a flow cytometer. A human or machine observer may use a variety of magnifying detection devices, such as a fluorescence microscope. The observer may optionally be capable of making an observation regarding whether an aliquot subsequently showed growth. In an embodiment a machine observer collects data, responsive to the data forms an image, e.g., a digital image, and assigns a value to the digital image, e.g., a value indicating the number of cells observed or whether growth is observed.

As used herein, the term “reliability of a single cell cloning technique” refers to how consistently a single cell cloning technique results in cell growth with a high probability of monoclonality.

As used herein, the term “interval” refers to a period when a single cell cloning technique is being practiced and no evaluation of a value probability of monoclonality is being performed. The period can be measured in aliquots formed, in containers comprising sets of aliquots filled, e.g., multi-well plates, e.g., 96-well plates, in time, or in other units known in the art.

As used herein, the term “threshold value of the probability of monoclonality” is a probability benchmark to which a calculated value of the probability of monoclonality can be compared. In some embodiments, a plurality of aliquots evaluated to have a value of probability of monoclonality that meets or exceeds a threshold value of the probability of monoclonality may proceed through a single cell cloning technique. In some embodiments, a plurality of aliquots evaluated to have a value of probability of monoclonality that is less than a threshold value of the probability of monoclonality may not proceed through a single cell cloning technique. In some embodiments, a threshold value of the probability of monoclonality is 0.95, 0.952, 0.954, 0.956, 0.958, 0.96, 0.962, 0.964, 0.968, 0.97, 0.972, 0.974, 0.976, 0.978, 0.98, 0.982, 0.984, 0.986, 0.988, 0.99, 0.992, 0.994, 0.996, 0.998, or 1. In some embodiments, a threshold value of the probability of monoclonality is 0.98. In some embodiments, a threshold value of the probability of monoclonality is 0.99.

As used herein, the term “endogenous” refers to any material from or naturally produced inside an organism, cell, tissue or system.

As used herein, the term “exogenous” refers to any material introduced to or produced outside of an organism, cell, tissue or system. Accordingly, “exogenous nucleic acid” refers to a nucleic acid that is introduced to or produced outside of an organism, cell, tissue or system. In an embodiment, sequences of the exogenous nucleic acid are not naturally produced, or cannot be naturally found, inside the organism, cell, tissue, or system that the exogenous nucleic acid is introduced into. Similarly, “exogenous polypeptide” refers to a polypeptide that is not naturally produced, or cannot be naturally found, inside the organism, cell, tissue, or system that the exogenous polypeptide is introduced to, e.g., by expression from an exogenous nucleic acid sequence.

As used herein, the term “heterologous” refers to any material from one species, when introduced to an organism, cell, tissue or system from a different species.

As used herein, the terms “nucleic acid,” “polynucleotide,” or “nucleic acid molecule” are used interchangeably and refers to deoxyribonucleic acid (DNA) or ribonucleic acid (RNA), or a combination of a DNA or RNA thereof, and polymers thereof in either single- or double-stranded form. The term “nucleic acid” includes, but is not limited to, a gene, cDNA, or an mRNA. In one embodiment, the nucleic acid molecule is synthetic (e.g., chemically synthesized or artificial) or recombinant. Unless specifically limited, the term encompasses molecules containing analogues or derivatives of natural nucleotides that have similar binding properties as the reference nucleic acid and are metabolized in a manner similar to naturally or non-naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others.

### Single Cell Cloning Techniques

One of the issues for consideration in the manufacture of a therapeutic protein is the requirement of a stable clonal cell line to ensure a consistent manufacturing process. The use of a non-clonal cell line may result in an uneconomical process or, even worse, variation in product quality and biological activity. Several single cell cloning techniques exist, including limited dilution single cell cloning (LDSCC), spotting (Clarke and Spier, 1980), capillary-aided cell cloning (Onadipe et al, 2001), and flow cytometry (e.g., fluorescence-activated cell sorting (FACS)), and each can be used with the methods disclosed herein.

Limited dilution single cell cloning involves diluting a culture into aliquots with a cellular concentration below one cell per aliquot, then culturing the aliquots to observe growth. Multiple rounds of time and labor intensive dilution and culturing are required to achieve monoclonality. The multiple rounds are required because LDSCC does not ensure that the growth observed, even after several rounds, is monoclonal.

Spotting is a technique involving separating a dilute solution of cells into 1 μl aliquots (e.g., droplets) using sterile Pasteur pipettes and depositing the droplets in a micro-well plate without touching the sides of the well, creating a free-standing aliquot that can be easily visually examined by an observer to determine the number of cells present. However, standard spotting protocols do not take into account the probability of an error in observer identification of cells in an aliquot. In some embodiments, the methods of the present disclosure can be applied to cell populations and aliquots produced in the application of a spotting technique. In some embodiments, the methods of the present disclosure evaluate the reliability of spotting-achieved monoclonality to ensure that any resultant cell line has a high probability of being monoclonal.

Capillary-aided cell cloning (CACC) is a technique similar to spotting, wherein separation of a solution of cells into approximately 1 μl aliquots (e.g. droplets) is achieved by using a capillary pipette, and examination of each droplet is carried out independently by two scientists. In some embodiments, the methods of the present disclosure can be applied to cell populations and aliquots produced in the application of a capillary-aided cell cloning (CACC) technique. In some embodiments, the methods of the present disclosure evaluate the reliability of CACC-achieved monoclonality to ensure that any resultant cell line has a high probability of being monoclonal.

Flow cytometry is a technique employing a device that flows a solution of cells through a narrow flow cell single file past a detector (e.g. a laser) coupled to a converter and computer, which can observe and process a characteristic of the cell. The flow cytometer can subsequently break the stream of cells into droplets (i.e. aliquots) containing on average less than one cell and deposit the aliquots into discrete addresses. Fluorescence-activated cells sorting (FACS) is a special application of flow cytometry that employs fluorescent dyes or fluorescent polypeptides on the surface of cells to identify cells to separate into discrete populations. However, standard protocols of single cell cloning employing flow cytometry do not take into account the probability of an error in observer (i.e. detector) identification of cells in an aliquot. In some embodiments, the methods of the present disclosure can be applied to cell populations and aliquots produced in the application of a flow cytometry technique. In some embodiments, the methods of the present disclosure, e.g., steps or algorithms described in the Examples, e.g., Example 11, may be adapted to accommodate a particular method of analysis, e.g., flow cytometry, e.g., FACS, machine or technique. In some embodiments, the methods of the present disclosure introduce controls that ensure that any resultant cell line has a high probability of being monoclonal.

In one aspect, the invention features a method of evaluating a value for probability of monoclonality, comprising: providing a solution comprising a population of cells; forming a plurality of aliquots of the solution; identifying aliquots having one cell; and providing, for aliquots identified as having one cell, a value for the probability that subsequent growth was monoclonal, thereby evaluating a value for probability of monoclonality. Through application of the methods disclosed herein to single cell cloning techniques, an assessment can be made of the likelihood that a growth proceeding from an aliquot is monoclonal, thus taking into account possible errors by observer(s).

In another aspect, the invention features a method of evaluating the reliability of a single cell cloning technique, comprising: a) providing a solution comprising a population of cells; b) performing a first estimate of the value of the probability of monoclonality of the single cell cloning technique, comprising: i) forming a plurality of aliquots of the solution; ii) identifying aliquots having one cell; and iii) providing, for aliquots identified as having one cell, a value of the probability that subsequent growth was monoclonal, c) practicing the single cell cloning technique for an interval, d) performing a second estimate of the value of the probability of monoclonality of the single cell cloning technique, comprising: i) forming a plurality of aliquots of the solution; ii) identifying aliquots having one cell; and iii) providing, for aliquots identified as having one cell, a value of the probability that subsequent growth was monoclonal; and e) comparing the first and second estimates of the value of the probability of monoclonality of the single cell cloning technique, thereby evaluating the reliability of a single cell cloning technique. By comparing values of the probability of monoclonality before and after practicing a single cell cloning technique, the reliability of the monoclonality of resultant cell growths can be evaluated. In an embodiment, drift, or a difference in the probability of monoclonality between the first and second estimates, can suggest adjustment of the parameters of the single cell cloning technique, e.g., to improve the probability of monoclonality. In an embodiment, c), d), and e) can be repeated for each interval of the single cell cloning technique, thereby providing evaluation of the reliability of the single cell cloning technique across multiple intervals.

In another aspect, the methods of the invention may be used to evaluate data from imaging systems or techniques, or in image processing software. In some embodiments, the methods may be applied to: body imaging, body scanners, whole body imaging, full body scanners, positron emission tomography (PET) scanning, PET/computed tomography (CT) scanning, magnetic resonance imaging, light microscopy, confocal microscopy, fluorescence microscopy, electron microscopy, cryo-electron microscopy, cryo-electron microscopy tomography, digital radiography imaging systems, digital fluoroscopy imaging systems, machine vision systems, live cell analyzers, fixed cell analyzers, high resolution imaging systems, high resolution cell imaging systems, laser scanner systems, and radioactive, fluorescent, or chemi-luminescent imaging systems.

### Applications for Production

The methods of evaluating a value for probability of monoclonality and methods of evaluating the reliability of a single cell cloning technique disclosed herein can be used to evaluate various cell lines or to evaluate the production of various cell lines for use in a bioreactor or processing vessel or tank, or, more generally with any feed source. The devices, facilities and methods described herein are suitable for culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products—such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermentor or fermentation unit, or any other reaction vessel and the term “reactor” is used interchangeably with “fermentor.” For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

In embodiments and unless stated otherwise herein, the devices, facilities, and methods described herein for use with methods of evaluating a value for probability of monoclonality can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

Methods described herein can be used for evaluating and producing monoclonal preparations of a broad spectrum cells. In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBv13.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii,), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B.megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the protein is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin,Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostim, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant Cl esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMlD, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In some embodiments, the polypeptide is adalimumab (HUMIRA), infliximab (REMICADE™), rituximab (RITUXAN™/MAB THERA™) etanercept (ENBREL™) bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 (adapted from US2016/0097074):

TABLE 1
Protein Products and Reference Listed Drug

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating | Leukine ®/Leukine ® |
| factor | Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone | |
| Antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell | RAPTIVA ™ |
| blocker | |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, | Synagis ™ |
| humanized mAb | |
| thyrotropin alfa | Thyrogen ® |
| Tenecteplase | TNKase ™ |
| Natalizumab | TYSABRI ® |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molecule, fusion protein, protein vaccine, or peptide as shown in Table 2, below.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, |
| | | Protropin, Siazen, Serostim, Valtropin |
| | Human follicle-stimulating | Gonal-F, Follistim |
| | hormone (FSH) | |
| | Human chorionic | Ovidrel |
| | gonadotropin | |
| | Lutropin-α | Luveris |
| | Glucagon | GlcaGen |
| | Growth hormone releasing | Geref |
| | hormone (GHRH) | |
| | Secretin | ChiRhoStim (human peptide), SecreFlo |
| | | (porcine peptide) |
| | Thyroid stimulating | Thyrogen |
| | hormone (TSH), thyrotropin | |
| Blood | Factor VIIa | NovoSeven |
| Clotting/Coagulation | Factor VIII | Bioclate, Helixate, Kogenate, |
| Factors | | Recombinate, ReFacto |
| | Factor IX | Benefix |
| | Antithrombin III (AT-III) | Thrombate III |
| | Protein C concentrate | Ceprotin |
| Cytokine/Growth | Type I alpha-interferon | Infergen |
| factor | Interferon-αn3 (IFNαn3) | Alferon N |
| | Interferon-β1a (rIFN- β) | Avonex, Rebif |
| | Interferon-β1b (rIFN- β) | Betaseron |
| | Interferon-γ1b (IFN γ) | Actimmune |
| | Aldesleukin (interleukin | Proleukin |
| | 2(IL2), epidermal | |
| | theymocyte activating | |
| | factor; ETAF | |
| | Palifermin (keratinocyte | Kepivance |
| | growth factor; KGF) | |
| | Becaplemin (platelet- | Regranex |
| | derived growth factor; | |
| | PDGF) | |
| | Anakinra (recombinant IL1 | Anril, Kineret |
| | antagonist) | |
| Antibody molecules | Bevacizumab (VEGFA | Avastin |
| | mAb) | |
| | Cetuximab (EGFR mAb) | Erbitux |
| | Panitumumab (EGFR mAb) | Vectibix |
| | Alemtuzumab (CD52 mAb) | Campath |
| | Rituximab (CD20 chimeric | Rituxan |
| | Ab) | |
| | Trastuzumab (HER2/Neu | Herceptin |
| | mAb) | |
| | Abatacept (CTLA Ab/Fc | Orencia |
| | fusion) | |
| | Adalimumab (TNFα mAb) | Humira |
| | Etanercept (TNF | Enbrel |
| | receptor/Fc fusion) | |
| | Infliximab (TNFα chimeric | Remicade |
| | mAb) | |
| | Alefacept (CD2 fusion | Amevive |
| | protein) | |
| | Efalizumab (CD11a mAb) | Raptiva |
| | Natalizumab (integrin α4 | Tysabri |
| | subunit mAb) | |
| | Eculizumab (C5mAb) | Soliris |
| | Muromonab-CD3 | Orthoclone, OKT3 |
| Other: | Insulin | Humulin, Novolin |
| Fusion | Hepatitis B surface antigen | Engerix, Recombivax HB |
| proteins/Protein | (HBsAg) | |
| vaccines/Peptides | HPV vaccine | Gardasil |
| | OspA | LYMErix |
| | Anti-Rhesus(Rh) | Rhophylac |
| | immunoglobulin G | |
| | Enfuvirtide | Fuzeon |
| | Spider silk, e.g., fibrion | QMONOS |

In embodiments, the protein is multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ®, | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, VEGFA | Blockade of 2 | Phase I | Solid tumors |
| (RO5520985, | | | proangiogenics | | |
| Roche) | | | | | |
| RG7716 (Roche) | CrossMab | Ang2, VEGFA | Blockade of 2 | Phase I | Wet AMD |
| | | | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG-fynomer | TNF, IL17A | Blockade of 2 | Phase I/II | Plaque psoriasis |
| | | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, has | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, | Blockade of 2 | Phase I | (Healthy |
| Serono, Ablynx) | | has | proinflammatory | | volunteers) |
| | | | cytokines, binds | | |
| | | | to HSA to | | |
| | | | increase half-life | | |
| ALX-0061 | Nanobody | IL-6R, has | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | has | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

### EXEMPLIFICATION

### Example 1: Capillary-Aided Cell Cloning

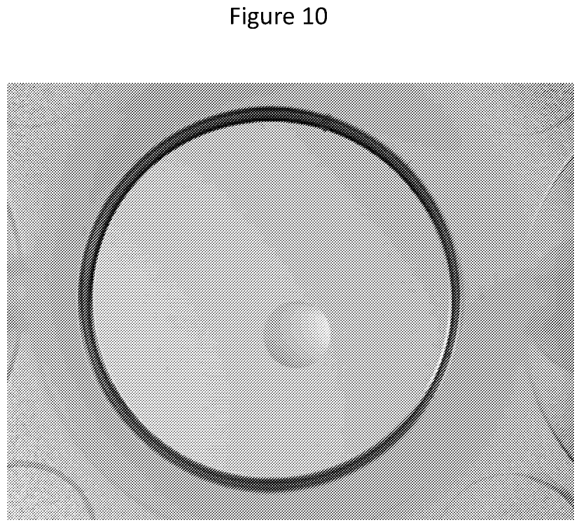

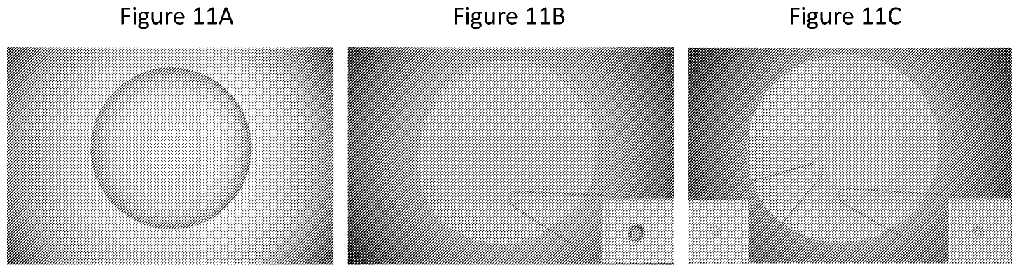

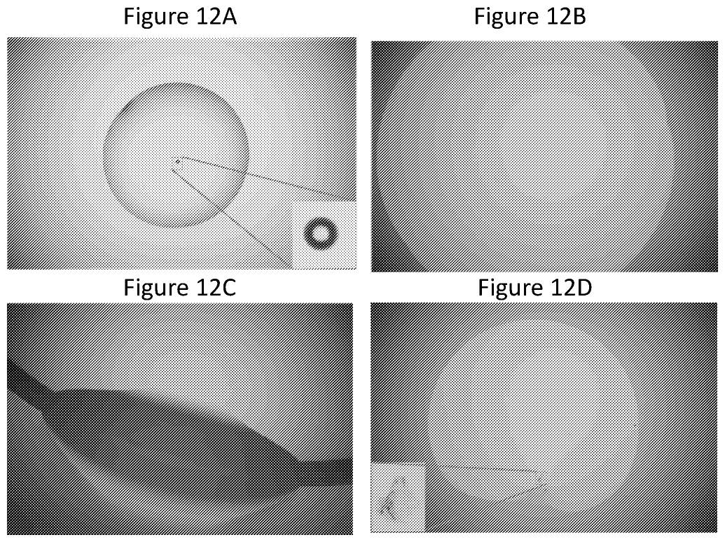

A cell count of the culture to be used for cloning was first performed. This culture was then diluted to approximately 1000 cells per ml. A droplet of approximately 1 μL of the diluted cell suspension was dispensed into 48-well plates (FIG. 10). Two scientists independently examined the droplets microscopically and recorded the number of cells contained (FIGS. 11A-11C). The observations were performed by initially scanning the whole droplet for the presence of cells at 40× magnification, then at 100× or 200× magnification to confirm the presence of only a single cell. Droplets that contained air bubbles, could not be completely visualized in a single field of view, for which the boundaries could not be clearly seen, or which contained debris were excluded from further analysis (FIGS. 12A-12D). After the observations, growth medium was added to all the wells. The plates were then incubated at 37° C. in an atmosphere containing 10% CO₂ and 90% air for up to 12 weeks, to allow for the growth of slow growing colonies. All the wells that produced colonies were recorded. Only colonies from wells containing one cell as agreed by both scientists were progressed.

### Example 2: Materials and Methods of Data Analysis

The observations of each of the scientists were summarised into three categories: no cells, one cell or more than one cell. The observed outcome for each well was that it showed either growth or no growth. This data was entered into a statistical model that was used to estimate the probability of monoclonality of the colonies using maximum likelihood. The calculation of the probability of monoclonality was performed using the software package, Mathematica version 4.1 (Wolfram Research, Inc.).

### Example 3: Validation of the Capillary-Aided Cell Cloning Technique

Possible errors in the visual observation made by the two scientists were considered. The first possible error was that that the two scientists may miss seeing a cell in the well and the presence of one cell when there were actually two cells. The second concern was that one cell could sit on top of another and the two cells can thus appear as one.

To address these concerns, an experiment was performed to validate the technique. In this experiment, two very similar GS-NS0 cell lines were mixed in the same proportion. The cell lines were derived from the same NS0 host cell bank and used the glutamine synthetase (GS) expression system to express similar antibodies that differed from each other only in minor changes in the variable region. In eleven separate sessions, four scientists seeded 2,300 wells with cells from the mixture of the two cell lines. The four scientists, working in pairs, confirmed that 321 of the 2300 wells seeded contained one cell each. After incubation for up to four weeks, growing colonies were found in 156 of these 321 wells. Validated ELISAs specific for each antibody showed that each of the 156 wells contained only one antibody. No wells were positive or negative for both antibodies (Table 1). These results indicated that the capillary-aided cell cloning technique resolved a mixed culture of two cell lines into monoclonal colonies. In this experiment, the error in the observations of the two scientists, based on cell growth in wells reported to contain no cells, were found to be very low at 0.4% (Table 2). This suggests that the chances of the two scientists missing the presence of a cell in a well were very low.

TABLE 4
Monoclonality of colonies obtained from a mixed culture
of two similar GS-NS0 cell lines producing different
antibodies after Capillary-Aided Cell Cloning

| Observation | Number of wells |
|---|---|
| Wells positive for antibody A | 94 |
| Wells positive for antibody B | 62 |
| Wells positive for both antibodies | 0 |
| Wells negative for both antibodies | 0 |
| Total | 156 |

TABLE 5
Quantification of the error associated with
the Capillary-Aided Cell Cloning technique

| Observation | Number of wells | |
|---|---|---|
| Wells scored as containing 0 cells | 474 | |
| Wells that subsequently showed growth | 2 | (0.4%) |
| Wells that subsequently showed no | 472 | (99.6%) |
| growth | | |

### Example 4: Developing a Mathematical Model

In the experiment described in Example 3, liquid containing a random distribution of cells, is dropped into a large number N of wells. Each well is then inspected independently by two scientists, who each have three options. They can report that the well contains no cells, one cell or more than one cell.

The observed outcome for each well is that it shows either growth, from one or more cells, or no growth. The latter may have resulted either because there was no cell in the well from which growth could start or because there were one or more cells but they did not grow. The result of such an experiment can be summarised by 12 frequencies nᵢⱼ where i indexes either growth (i=1) or no growth (i=0), j indexes the six combinations of reports from the two scientists and nᵢⱼ denotes the number of wells that fall into the category (i,j). It is implicitly assumed that the two scientists are not identified. If the experiment records which scientist makes which report, and only two or a few scientists are used, then a different model to the one specified below should be used. The following table should illustrate all key concepts.

TABLE 6
An illustration of all key concepts

| | | No. of wells | No. of wells |
|---|---|---|---|
| | | with no growth | with growth |
| j= | Scientists' reports | (i = 0) | (i = 1) |
| 1 | Both say no cells | n₀₁ | n₁₁ |
| 2 | One says no cells, the other | n₀₂ | n₁₂ |
| | says one cell | | |
| 3 | Both say one cell | n₀₃ | n₁₃ |
| 4 | One says no cells, the other | n₀₄ | n₁₄ |
| | says more than one cell | | |
| 5 | One says one cell, the other | n₀₅ | n₁₅ |
| | says more than one cell | | |
| 6 | Both say more than one cell | n₀₆ | n₁₆ |

If a well shows growth, this may have arisen from just one cell, and so be monoclonal, or it may be a mixture of growths from two or more cells. If the scientists are skilled, the best chance of finding monoclonal growth is amongst the n₁₃ wells for which both scientists report there was initially just one cell present and which subsequently showed growth. It is therefore required to estimate the proportion P of these wells that do, in fact, have monoclonal growth.

It has to be noted that this quantity is not directly observable and an estimate of it has to be inferred from the experimental data.

In all experiments in which an unobservable quantity has to be estimated, the estimate has to be based on a set of assumptions, and the validity of the estimate stands or falls by the reasonableness of the assumptions. Here the following set of assumptions have been made:
1. The actual number of cells initially in a well follows a Poisson distribution with unknown mean μ. The numbers in different wells are independently and randomly drawn from this distribution, and the expected or average number in a well is the same for all wells.
2. Each cell has the same unknown probability p of growing, independently of all other cells and of how many cells are in the same well.
3. A well shows growth if and only if one or more cells in that well grow.
4. When there are actually k cells in a well, the probability that the scientists report combination j is an unknown quantity πₖⱼ. For each value of k the sum of these over j=1 to 6 has to be 1.

From assumption 1, the probability that a well contains k cells is e^{-μ}μ^{k}/k!, where k=0, 1, 2, 3, . . .

From assumptions 2 and 3, the probability that a well containing k cells shows no growth is (1−p)^{k}.

If pᵢⱼ denotes the probability that any well falls into the combination (i,j) in Table 3 showing the different possible outcomes, the formulae for all 12 of these can now be derived using assumption 4. For example:

$$\begin{matrix}{p}_{01} = prob ( no growth \underline{and} both scientists say no cells ) \\ = {\sum }_{k = 0}^{∞} prob ( k cells present ) prob ( no growth \vert k cells ) prob \\ ( both scientists say no cells \vert k cells ) \\ = {\sum }_{k = 0}^{∞} {e}^{- μ} {μ}^{k} \text{/} k ! {( 1 - p )}^{k} {π}_{k 1}\end{matrix}$$

$$and$$

$${p}_{11} = {\sum }_{k = 0}^{∞} {e}^{- μ} {μ}^{k} \text{/} k ! [ 1 - {( 1 - p )}^{k} ] {π}_{k 1}$$

There are five more similar pairs of equations with the second subscript on the p's and π's changing from 1 through to 6.

The model has so far introduced an infinite number of unknown quantities. These are μ, p and all the πₖⱼ with j=1 to 6 and k=0, 1, 2, 3, 4, . . . . Such a model cannot fail to provide an exact fit to any set of data, and sensible conditions must be imposed to restrict the number of unknowns before a usable model can be obtained. There are, of course, many ways of doing this but as a first step assumption 4 above can be replaced by
5. When there are actually k cells in a well, each scientist independently has probability qₖₘ of reporting no cells (m=0), one cell (m=1) or more than one cell (m=2), with qₖ₀+qₖ₁+qₖ₂=1.

This has the effect of replacing each set of five unknown π's (six subject to the constraint that they must add up to 1) by a set of two unknown q's. The relation between them is given simply by the equations:

πₖ₁=qₖ₀²

πₖ₂=2qₖ₀qₖ₁

πₖ₃=qₖ₁²

πₖ₄=2qₖ₀qₖ₂

πₖ₅=2qₖ₁qₖ₂

πₖ₆=qₖ₂²

There are still, however, an infinite number of such sets, so further restrictions are needed. The following assumptions are proposed initially. They put into symbols the notion that both scientists are reasonably competent and do not make big mistakes.
6. When there are 3 or more cells in a well, each scientist is certain to report “more than one cell”.
7. When there are 2 or more cells in a well, each scientist is certain not to report “no cells”.

The remaining unknown q's can be put schematically into a table where asterisks indicate non-zero probabilities but constrained to make each column total 1:

TABLE 7
Reducing the number of unknown q's

Actual number of cells

| Report | 0 | 1 | 2 | ≥3 |
|---|---|---|---|---|
| No cells | q₀₀ | q₁₀ | 0 | 0 |
| One cell | q₀₁ | q₁₁ | q₂₁ | 0 |
| More than one cell | * | * | * | 1 |

Therefore, now there are only 5 unknown q's, making 7 unknowns in all. This should enable a good fit to the 12 observed frequencies nᵢⱼ provided the model is a reasonable representation of reality.

There is one further constraint, namely that q₂₁ should be at least as big as q₁₀. This is because it is possible that when there are actually two cells present, one can almost completely obscure the other, making it look as if only one is present. It is felt that this error is more likely to occur than the other kind of error, of not seeing one cell when there is actually one cell present.

### Example 5: Criteria for Goodness of Fit of the Model to Data

Maximum Likelihood

The likelihood is simply the probability that we would have observed what we did observe if the model had been true. It is a function not only of the observed data but also of the unknown parameters in the model. We naturally wish to choose those values of the unknown parameters which maximise the likelihood because these, in a primitive sense, best “explain” how come we observed what we did observe. In our case, therefore, we think of the likelihood as a surface in 7 dimensions and we seek to find the “summit” of this surface.

The formula for the likelihood is simply the product of all of the probabilities of the outcomes for each one of the N wells. This can be written as

$$L = {\prod }_{i = 0}^{1} {\prod }_{j = 1}^{6} {p}_{ij}^{{n}_{ij}} . \\ Minimum sum of squares$$

For each observed frequency nᵢⱼ we can calculate the expected frequency eᵢⱼ predicted by the model. We might try to find the values of the unknown parameters in the model which minimise the sum of squares of the discrepancies between observed and expected frequencies. This is given by

$$S = {\sum }_{i = 0}^{1} {\sum }_{j = 1}^{6} {( {n}_{ij} - {e}_{ij} )}^{2}$$

$$Minimum chi \text{-} square$$

As a variation on the sum of squares above, we might wish to weight each squared discrepancy between observed and expected frequency inversely by the expected frequency, the idea being that the difference between an observed frequency of 1002 and an expected one of 1000 is less “serious” than the difference between 102 and 100 or between 12 and 10. The familiar chi-square statistic achieves this in what is, in many senses, an optimal way. It is given by

$$C = {\sum }_{i = 0}^{1} {\sum }_{j = 1}^{6} {( {n}_{ij} - {e}_{ij} )}^{2} \text{/} {e}_{ij}$$

$$Log likelihood ratio statistic$$

An alternative measure of overall discrepancy which is often used is given by

$$G = 2 {\sum }_{i = 0}^{1} {\sum }_{j = 1}^{6} {n}_{ij} log ( {n}_{ij} / {e}_{ij} )$$

All four quantities L, S, C and G are complicated functions of the 7 unknown parameters. We seek the maximum value of L, or equivalently of log L (this will typically be a large negative number) but the minimum values of S, C and G. There is no way this can feasibly be done algebraically by differentiation, so one of the many function maximisation algorithms must be used. These all suffer from a major disadvantage, namely that they require some initial guesses at the values of the unknowns to use as a starting point for their sequential search routines. Even worse, the answer they finally produce may well depend on the starting values they are given. If the 7-dimensional surface of, say, L as a function of the 7 unknowns is smooth and has a single peak then there is usually no problem and the routines will find the peak regardless of the starting values, but if the surface is more like a mountain range with peaks of different heights in different places then the routines can easily get side-tracked into finding a minor peak and stopping without noticing that there is an even higher peak somewhere else. There are only two ways of guarding against this:
carefully choosing starting values which are as good as prior knowledge permits. There should be considerable information in advance, particularly about the values of μ, and p, and this should be used.
carefully inspecting the answers to see if they are biologically sensible.

Even when all seems clear and correct, confirmation should be obtained by running several other sets of starting values quite close to the initial set and checking that the answers they produce are essentially the same.

One other complication needs to be mentioned. The quantities L, S, C and G are defined only over a limited range of values of the 7 unknowns. The constraint

0≤p≤1

is obvious enough, but there are others, such as

0≤q₀₀≤1

0≤q₀₁≤1−q₀₀

which need to be carefully programmed into the numerical routines. The peaks may well occur very close to some of the boundaries of the permissible region which can again cause problems with the convergence of the calculations towards the final answer.

In summary, this model will never be a means of mindlessly feeding in a set of experimental data and obtaining a guaranteed-correct answer. It must always be used with care and the answers viewed sceptically until confirmation is obtained.

### Example 6: Estimating the Probability of Monoclonality

All of this modelling and fitting of the model to the data has one main purpose. This is to estimate the probability that, if a well is reported to contain exactly one cell by both scientists and if the well subsequently shows growth, then that growth will in fact be monoclonal. This is given by:

$$\begin{matrix}P = prob ( \begin{matrix}monoclonal given both scientists \\ report 1 cell and growth occurs\end{matrix} ) \\ = \frac{prob ( monoclonal and both report 1 cell and growth )}{prob ( both report 1 cell and growth )}\end{matrix}$$

The numerator can be written as

$${\sum }_{k = 1} prob ( \begin{matrix}monoclonal and both report 1 cell and \\ growth given k cells\end{matrix} ) prob ( k cells ) = {\sum }_{k = 1} {kp ( 1 - p )}^{k - 1} {q}_{k 1}^{2} {e}^{- μ} {µ}^{k} \text{/} k \text{!} = p {q}_{11}^{2} µ {e}^{- µ} + 2 p ( 1 - p ) {q}_{21}^{2} {µ}^{2} {e}^{- µ} \text{/} 2$$

and the denominator as

$${\sum }_{k = 1} prob ( both report 1 cell and growth given k cells ) prob ( k cells ) = {\sum }_{k = 1} [ 1 - {( 1 - p )}^{k} ] {q}_{k 1}^{2} {e}^{- µ} {µ}^{k} \text{/} k \text{!} = p {q}_{11}^{2} µ {e}^{- µ} + ( 2 p - {p}^{2} ) {q}_{21}^{2} {µ}^{2} {e}^{- µ} \text{/} 2$$

so the ratio becomes, after simplification,

$$P = \frac{2 {q}_{11}^{2} + 2 ( 1 - p ) {q}_{21}^{2} μ}{2 {q}_{11}^{2} + ( 2 - p ) {q}_{21}^{2} μ} .$$

The values of the unknowns estimated by the numerical processes in the previous section, therefore, have to be inserted into this formula to obtain the estimated value of P.

### Example 7: Assumptions about Scientist Skill

The major limitation of the model is that the two scientists are assumed to be equally skillful, in that they are assumed to have the same chances of making the three possible reports. If the scientists are not identifiable, there seems to be no way of improving on this. If, however, the whole experiment was done using identified scientists, labelled 1 and 2, say, then it would convey much more information. The outcome “one scientist reports no cell, the other reports one cell”, for example, could be divided into two “scientist 1 reports no cell, scientist 2 reports one cell” and “scientist 2 reports no cell, scientist 1 reports one cell”. We could introduce different sets of q's for each scientist to allow for their different skills.

### Example 8: Numerical Example

Results from an experiment dating from about 1996 are given below.

TABLE 8
Numerical Example Observer Data

| | Number of wells | Number of wells |
|---|---|---|
| | with no growth | with growth |
| Scientists' reports | (i = 0) | (i = 1) |
| Both say no cells | 472 | 2 |
| One says no cells, the other | 96 | 17 |
| says one cell | | |
| Both say one cell | 144 | 177 |
| One says no cells, the other | 29 | 1 |
| says more than one cell | | |
| One says one cell, the other | 39 | 52 |
| says more than one cell | | |
| Both say more than one cell | 101 | 375 |

Initial guesses at values for the unknowns were μ=0.4, p=0.25 and

TABLE 9
Numerical Example Initial Values 1

| | Number of cells in a well | | | |
|---|---|---|---|---|
| Report | 0 | 1 | 2 | ≥3 |
| No cells | q₀₀ = 0.85 | q₁₀ = 0.10 | 0 | 0 |
| One cell | q₀₁ = 0.13 | q₁₁ = 0.80 | q₂₁ = 0.15 | 0 |
| More than one cell | * = 0.02 | * = 0.10 | * = 0.85 | 1 |

where the asterisked values are supplied by default to make each column add up to 1.

The estimates of μ and p from the maximum likelihood criterion were

μ=1.0909, p=0.5083

and the estimates of the q's were

TABLE 10
Numerical Example Estimated Values 1

| | Number of cells in a well | | | |
|---|---|---|---|---|
| Report | 0 | 1 | 2 | ≥3 |
| No cells | q₀₀ = 0.9106 | q₁₀ = 0.0489 | 0 | 0 |
| One cell | q₀₁ = 0.0648 | q₁₁ = 0.8551 | q₂₁ = 0.0489 | 0 |
| More than one cell | * = 0.0246 | * = 0.0960 | * = 0.9511 | 1 |

If these answers are correct, the scientists were even more skilled than we gave them credit for in our initial estimates.

The estimated probability of monoclonality P was 0.9991.

In order to check the internal validity of the modelling process, we can work out what frequencies we should have expected to see in each of the twelve categories. Those derived from the maximum likelihood criterion are inserted in brackets to accompany each corresponding observed frequency.

TABLE 11
Numerical Example Observer vs. Expected Data

| | Observed (Expected) | | Observed (Expected) | |
|---|---|---|---|---|
| | number with no growth | | number with growth | |
| Scientists' reports | (i = 0) | | (i = 1) | |
| Both say no cells | 472 | (419.89) | 2 | (0.67) |
| One says no cells, the | 96 | (82.33) | 17 | (23.45) |
| other says one cell | | | | |
| Both say one cell | 144 | (200.56) | 177 | (205.51) |
| One says no cells, the | 29 | (25.18) | 1 | (2.63) |
| other says more than | | | | |
| one cell | | | | |
| One says one cell, the | 39 | (52.90) | 52 | (67.25) |
| other says more than | | | | |
| one cell | | | | |
| Both say more than | 101 | (83.54) | 375 | (341.07) |
| one cell | | | | |

In order to show the effects of inappropriate choice of starting values, the analyses were run again using a different set of starting values, with μ=0.4 and p=0.25 as before but

TABLE 12
Numerical Example Initial Values 2

| | Number of cells in a well | | | |
|---|---|---|---|---|
| Report | 0 | 1 | 2 | ≥3 |
| No cells | q₀₀ = 0.40 | q₁₀ = 0.40 | q₂₀ = 0 | 0 |
| One cell | q₀₁ = 0.40 | q₁₁ = 0.40 | q₂₁ = 0.40 | 0 |
| More than one cell | * = 0.20 | * = 0.20 | * = 0.60 | 1 |

The results were

μ=1.0915, p=0.5081, P=0.9991

and the estimates of the q's were

TABLE 13
Numerical Example Estimated Values 2

| | Number of cells in a well | | | |
|---|---|---|---|---|
| Report | 0 | 1 | 2 | ≥3 |
| No cells | q₀₀ = 0.9108 | q₁₀ = 0.0490 | 0 | 0 |
| One cell | q₀₁ = 0.0647 | q₁₁ = 0.8552 | q₂₁ = 0.0490 | 0 |
| More than one cell | * = 0.0246 | * = 0.0958 | * = 0.9510 | 1 |

These are the same as before, with small differences in the fourth decimal place. Six other sets of starting values were tried and five of them converged to the same solution as above. The one exception converged to a solution that was clearly wrong. The peak of the likelihood surface which it found was well below the peak found by the other solutions, and the q's were inappropriate. It is instructive, though, that the starting values which produced this wrong answer were

μ=1.1553, p=0.5514

with the same set of 0.4 values for the q's as above. The starting values for μ and p are ones produced by using crude estimates from the raw data and were, in fact, very close to the “correct” values found by the other solution. Starting with “good” initial values for some unknowns is therefore no guarantee of getting the best answer.

It should also be noted that the estimate of q₂₁ always came out to be exactly the same as that of q₁₀. It would have been lower but for the constraint q₂₁≥q₁₀ and this would have had the effect of making the probability of monoclonality P even closer to 1.

This example shows that results must always be examined critically. In most cases quite a lot of sets of starting values will probably be needed before any one set of answers can be accepted with comfort.

### Example 9: Adapting the Model for Mathematica

The procedure for estimating the starting values for μ and p was modified to allow Mathematica to calculate these values from the data supplied from the cloning experiments. The starting value of μ¹ can be roughly estimated from the total number of wells seeded and calculating the average number of cells seeded per well based on the observations reported by the two scientists. The starting value of p² can be roughly estimated from the ratio of the number of wells that show growth by the total number of the wells in the category where the two scientists reported the presence of one cell. It was considered that a better estimate of the starting values for μ and p can be obtained in this way. While the results were similar whether initial values were given for μ and p or not, in practice, no initial values will be given for μ and p. ¹−ln[(n₀₁+n₁₁)/N]² n₁₃/(n₀₃+n₁₃)

As the probability of clonality was very close to 1, an estimate of the 95% lower bound of the probability of monoclonality (P) was thought to be the most practical way to determine how good an estimate the probability was. This was performed by taking the natural logarithm of the ratio of the estimates of P and 1−P, and invoking the common assumption that its distribution is approximately normal.

### Example 10: Applying the Model to Capillary-Aided Cell Cloning of Cell Lines

The mathematical model was applied to data obtained from the cloning of several cell lines performed using the capillary aided cell cloning technique. The probability of monoclonality obtained from 24 clonings to date was 0.9827 to 0.9999. This shows that the capillary aided cell cloning technique is a reliable one-step method for cloning to achieve a high probability of monoclonality.

TABLE 14
Probability of monoclonality of cell lines
derived using Capillary-aided Cell Cloning.

| | | Probability of |
|---|---|---|
| Cell Line | Cell Type | monoclonality |
| A | NS0 | 0.9998 |
| B | NS0 | 0.9997 |
| C | NS0 | 0.9998 |
| D | NS0 | 0.9987 |
| E | NS0 | 0.9885 |
| F | NS0 | 0.9961 |
| G | NS0 | 0.9986 |
| H | NS0 | 0.9957 |
| I | NS0 | 0.9987 |
| J | NS0 | 0.9999 |
| K | NS0 | 0.9998 |
| L | CHO | 0.9827 |
| M | CHO | 0.9915 |
| N | CHO | o.9976 |
| O | CHO | o.9983 |
| P | CHO | 0.9955 |
| Q | HYBRIDOMA | 0.9997 |
| R | NS0 | 0.9998 |
| S | NS0 | 0.9997 |
| T | NS0 | 0.9997 |
| U | NS0 | 0.9997 |
| V | NS0 | 0.9966 |
| W | NS0 | 0.9995 |
| X | NS0 | 0.9995 |

One round of capillary-aided cell cloning can replace two rounds of limiting dilution cloning to obtain a monoclonal cell line. The technique can be used routinely to demonstrate monoclonality.

The model developed is robust and predicts results that show good agreement with experimental data. The use of this model and the data presented provide sufficient data to support the method. The model permits the estimation of the probability of monoclonality and an estimate of the 95% lower bound for this probability can also be calculated.

### Example 11: Improving FACS Based Single Cell Cloning

Gaps in current FACS set-ups were identified and controls were introduced that ensure that any resultant cell line has a high probability of being monoclonal. An experiment was devised to show how a reproducible high probability of monoclonality (≥0.99) has been achieved using FACS. Following careful instrument set-up, a representative sample of cells is fluorescently stained and single cell sorted, onto a first 96-well plate-lid, using a series of gates to exclude cell debris, non-viable cells and cell aggregates. These 96-well plate-lids are visually inspected using fluorescence microscopy. At least one scientist inspects the aliquots in the wells in the image, make observations of 0 cells, 1 cell or ≥2 cells, and the observations are recorded. The number of observations for each category is used to estimate the probability of monoclonality using a probability equation, e.g., the equation developed in Example 6 or a similar equation that uses prior to posterior Bayesian analysis. Since use of the FACS assumes each droplet contains a cell, statistical methods based upon random distribution of cells in the droplets are not appropriate. After an initial assessment of the reliability of monoclonality has been made of a first 96-well plate lid, a further 1, 5, 10, 15, 20, 25, or more plates are filled with aliquots of a population of unstained cells selected for cloning using FACS. After this interval, a second 96-well plate lid is inspected using fluorescence microscopy. Again at least one scientist inspects the aliquots in the wells in the image, make observations of 0 cells, 1 cell or ≥2 cells, and the observations are recorded, and again the number of observations for each category is used to estimate the probability of monoclonality. If the second estimate of the probability of monoclonality is altered from the first estimate or does not meet or exceed a threshold probability of monoclonality, the plates of the preceding interval will not be progressed further. If instrument performance drifts, appropriate control strategies are used to return the FACS to its desired performance envelope. Use of such control strategies increases the confidence that a well contains a single cell. With this increased control over the method, the utility and reliability of FACS for generating cell lines for bioproces sing uses greatly increases.

More specific details for using FACS based single cell cloning are described below. The steps and/or algorithm used may be adapted to the machine-specific characteristics of a particular flow cytometer, e.g., FACS, machine or technique.

### Instrument Set-Up

Prepare for aseptic sort
   Replacement of all disposable flow path tubing and sanitisation of instrument according to manufacturer's guidelines
   Confirmation of sheath fluid and stream sterility
Stabilisation of stream
   Checks performed to ensure consistent behaviour of stream
   Fixed parameters established for stream frequency, drop break-off and gap field
   Confirmation of stable side stream formation
Cytometer performance check
   Use beads to set PMT gains
   Check instrument performance using control chart
Laser alignment and area scaling factor check
   Performance verification of cytometer channels
   Verification of area scaling factor
Deposition and drop delay setting
   Deposition position from the sort stream is confirmed/adjusted for centre of well
   Optimise drop delay to achieve maximum yield in the sort stream
Confirmation of set-up using beads
   Sort fluorescent beads using a single cell precision mask
   Visually confirm deposition of 1 bead per well; if more than 1 bead present in any well the set-up is repeated
Confirmation of set-up using representative cells
   Establish cell population within gates that exclude debris and cell aggregates
   Sort fluorescently stained representative cells using a single cell precision mask
   Visually confirm deposition of 1 cell per well; calculated probability of monoclonality must be ≥0.99 otherwise repeat set-up
System ready for sorting
   All parameter voltages and gates are fixed
   Any changes to system setting require reconfirmation of set-up using cells

### Gating Strategy and Single Cell Sorting

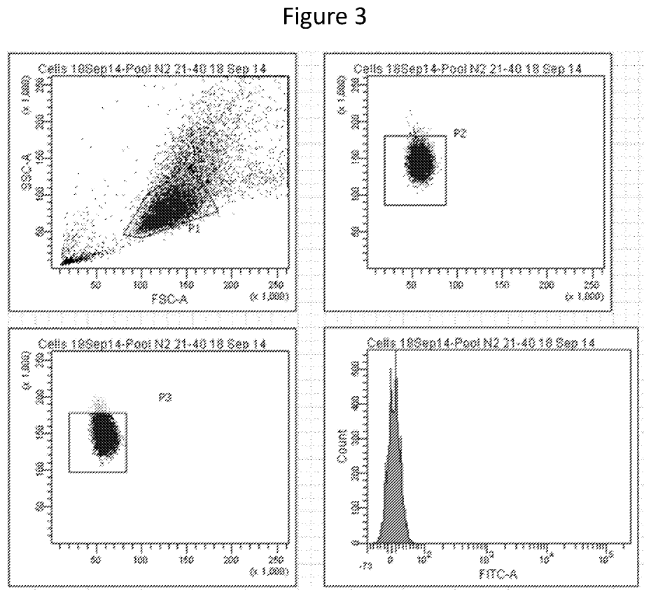

Fresh cell populations were prepared for each sorting session which included passing cells through a cell filter to break up any cell aggregates. The cells were then subjected to a gating strategy which excluded non-viable cells, debris and remaining doublets or higher order cell aggregates as shown in FIG. 3. Fluorescence is not used to aid in identification and selection of cells for sorting.

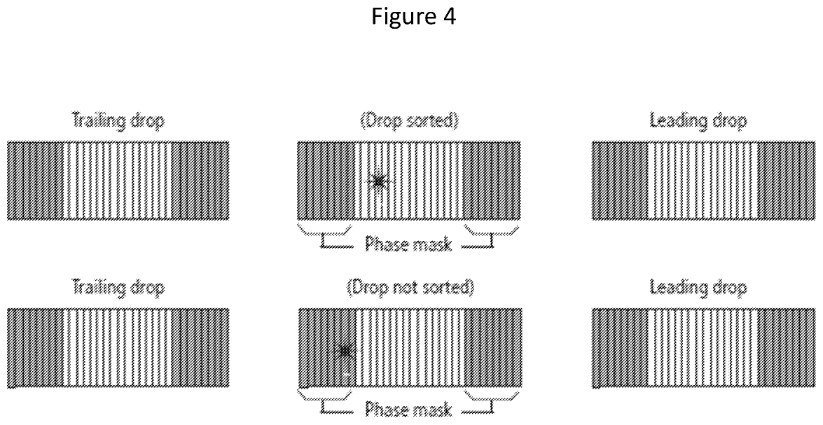

Cells from the selected population were single-cell sorted into multi-well plates (typically 20×96-well plates per sort session) using a single cell precision mask. The droplet containing a cell was only sorted if the droplet was free of contaminating particles and was centred within the droplet (FIG. 4). The leading and training droplets were not sorted. This allowed for high purity of sorted droplets although a large proportion of cells were discarded to waste.

### Measuring Consistency of Instrument Performance

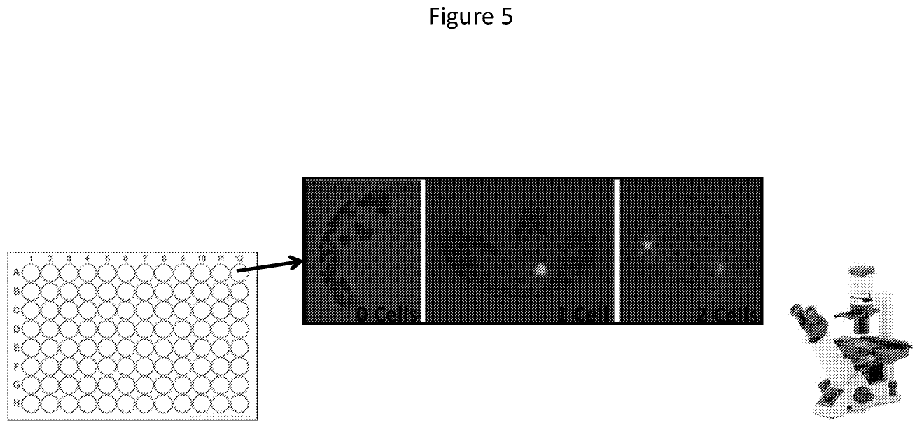

The instrument performance was measured at regular intervals by staining a cell population with ER-Tracker™ Green (Life Technologies) to aid in visual identification of the cell population followed by sorting onto the lid of a 96-well plate as a target. The markings on the lid corresponded to the position of the well in a 96-well plate. The droplets on the plate lid were then manually checked using a fluorescent microscope and the number of cells in each target was recorded as either 0, 1, or 2+ cells (FIG. 5). This process was repeated at the beginning and end of each sort session and the resulting data set was used to calculate the probability of monoclonality for the sort session. If the probability of monoclonality was calculated to be <0.990 at the start and/or end of a cloning session the instrument was not considered suitable for single-cell deposition and the appropriate corrective action was implemented. The plates sorted during the encompassing session were discarded and the instrument set-up confirmation using cells was repeated. Likewise if the instrument behaviour was not considered consistent over the course of a sort session (e.g. cell deposition position within the plate shifts), this would also trigger corrective action and reconfirmation of the instrument set-up as above.

### Statistical Model for Calculating Probability of Monoclonality

The probability (P) that a target has zero (X=0) or a single cell (X=1) was estimated using a prior to posterior Bayesian analysis. The probability of monoclonality was estimated as:

$$P ( monoclonality ) = \frac{P ( X = 1 )}{1 - P ( X = 0 )}$$

This is equivalent to the expression S/R, where S=the number of wells containing single colonies and R=the number of wells responding (i.e. growing) which is frequently used in limiting dilution cloning (Coller & Coller, Hybridoma, 2(1):91-96, 1983).

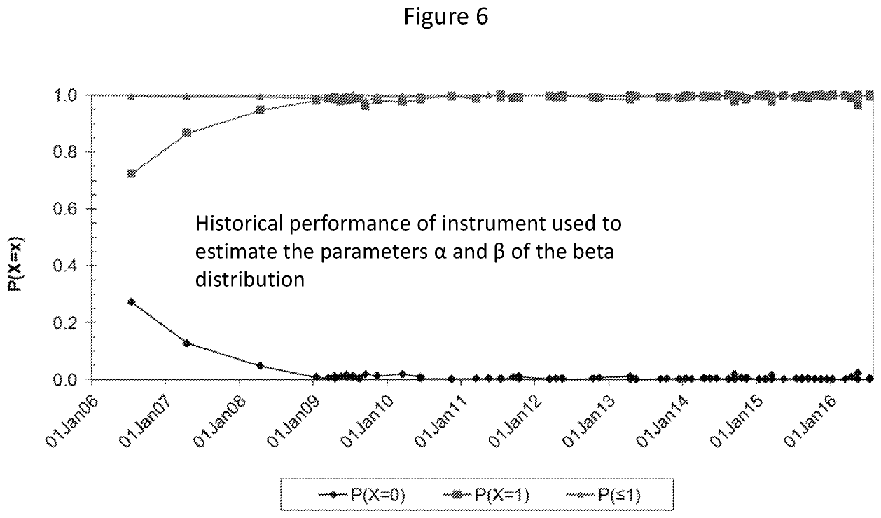

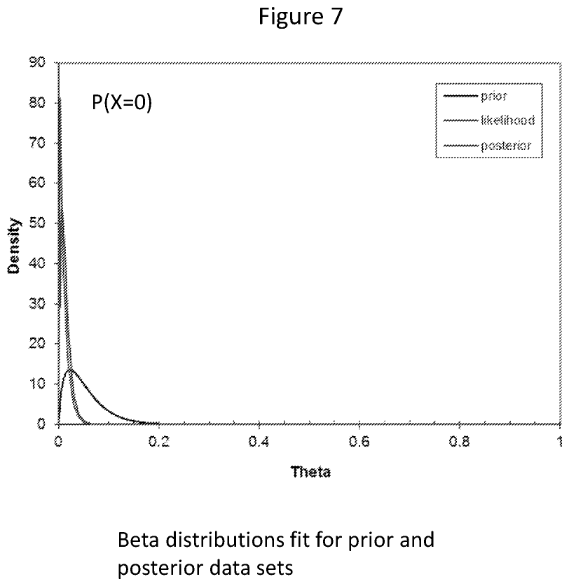

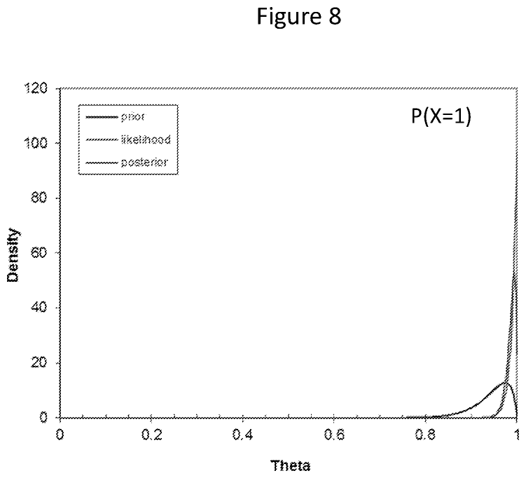

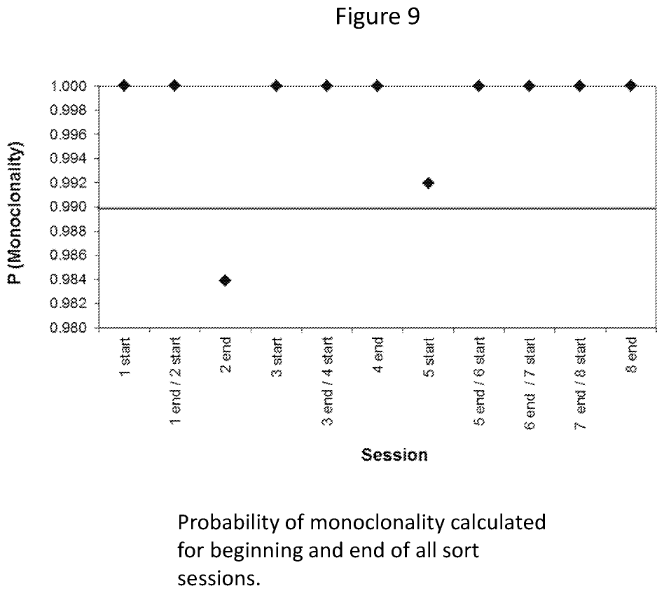

For a FACS operated in single cell sort mode, nearly all droplets will contain cells thus violating the assumption of randomness that underpins the Poisson distribution. A Bayesian approach is therefore used to estimate P(X=0) and P(X=1) because no assumption of the underpinning distribution is needed. The Bayesian model uses the previous performance of the instrument (FIG. 6) to predict the outcome of the sampled data. A beta distribution is used as the conjugate prior and posterior (FIGS. 7 and 8). The values for P(X=0) and P(X=1) were estimated as the mode of the posterior distribution (FIG. 9).

### CONCLUSIONS

FACS can be used to isolate single cells with a high probability of monoclonality (≥0.990) through use of robust instrument set-up and regular monitoring of instrument performance. A Bayesian model can be applied to estimate a probability of monoclonality for each single-cell sorting session based on previous performance of the FACS instrument. Such a FACS-assisted single cloning round can reduce the time and cost of developing a cell line suitable for manufacturing biotherapeutics. Further assurance of monoclonality can be provided through single cell imaging and/or monitoring of colony outgrowth.

We claim:

1. A method of evaluating a value for probability of monoclonality, comprising:
a) providing a solution comprising a population of cells;
b) forming a plurality of aliquots of the solution;
c) identifying aliquots having one cell; and
d) providing, for aliquots identified as having one cell, a value for the probability that subsequent growth was monoclonal,
thereby evaluating a value for probability of monoclonality.

2. The method of claim 1, wherein b) comprises:
b) forming a plurality of aliquots of the solution: with a printing device, by pipetting, using a capillary device (e.g., as in CACC), or using fluorescence-activated cell sorting (FACS) or flow cytometry.

3. The method of either claim 1 or 2, wherein b) comprises:
b) forming a plurality of aliquots of the solution using a capillary device (e.g., by CACC).

4. The method of any of claim 1 or 2, wherein b) comprises:
b) using fluorescence-activated cell sorting (FACS) or flow cytometry to form a plurality of aliquots of the solution.

5. The method of any of claims 1-4, wherein c) comprises a plurality of observers, identifying aliquots as having one cell and showing subsequent growth.

6. The method of any of claims 1-5, wherein c) comprises two observers identifying aliquots as having one cell and showing subsequent growth.

7. The method of any of claims 1-6, wherein c) comprises two observers identifying whether an aliquot has zero, one, or more cells.

8. The method of any of claims 1-7, wherein c) comprises two observers identifying whether an aliquot has zero, one, or more cells, and identifying whether an aliquot shows subsequent growth.

9. The method of any of claims 1-8, wherein d) comprises:
i) calculating data values for the frequencies at which aliquots were identified as having zero, one, or more cells, and whether the aliquots showed or did not show subsequent growth; and
ii) using a probability equation and the data values to evaluate the probability that the subsequent growth of an aliquot identified as having one cell is monoclonal.

10. The method of any of claims 1-9, wherein d) i) comprises:
i) calculating data values for the frequencies at which aliquots were identified as having zero, one, or more cells, and whether the aliquots showed or did not show subsequent growth, the data values comprising the data values listed in Table 6.

11. The method of any of claims 1-10, wherein d) i) comprises:
i) calculating data values for the frequencies at which aliquots were identified as having zero, one, or more cells, and whether the aliquots showed or did not show subsequent growth, the data values comprising:
n₀₁, the number of aliquots two observers identified as containing zero cells that did not show subsequent growth;
n₀₂, the number of aliquots one observer identified as containing zero cells and one observer identified as containing one cell that did not show subsequent growth;
n₀₃, the number of aliquots two observers identified as containing one cell that did not show subsequent growth;
n₀₄, the number of aliquots one observer identified as containing zero cells and one observer identified as containing more than one cell that did not show subsequent growth;
n₀₅, the number of aliquots one observer identified as containing one cell and one observer identified as containing more than one cell that did not show subsequent growth;
n₀₆, the number of aliquots two observers identified as containing more than one cell that did not show subsequent growth;
n₁₁, the number of aliquots two observers identified as containing zero cells that showed subsequent growth;
n₁₂, the number of aliquots one observer identified as containing zero cells and one observer identified as containing one cell that showed subsequent growth;
n₁₃, the number of aliquots two observers identified as containing one cell that showed subsequent growth;
n₁₄, the number of aliquots one observer identified as containing zero cells and one observer identified as containing more than one cell that showed subsequent growth;
n₁₅, the number of aliquots one observer identified as containing one cell and one observer identified as containing more than one cell that showed subsequent growth; and
n₁₆, the number of aliquots two observers identified as containing more than one cell that showed subsequent growth.

12. The method of any of claims 1-11, wherein d) ii) comprises:
ii) fitting/applying the data values to a probability equation comprising unknowns consisting of the parameters listed in Table 6 to evaluate the probability that the subsequent growth of an aliquot identified as having one cell is monoclonal.

13. The method of any of claims 1-12, wherein d) ii) comprises:
ii) fitting/applying the data values to a probability equation comprising unknowns consisting of:
q₀₀, the probability of an observer identifying an aliquot as containing zero cells when the aliquot actually contains zero cells;
q₁₀, the probability of an observer identifying an aliquot as containing zero cells when the aliquot actually contains one cell;
q₀₁, the probability of an observer identifying an aliquot as containing one cell when the aliquot actually contains zero cells;
q₁₁, the probability of an observer identifying an aliquot as containing one cell when the aliquot actually contains one cell;
q₂₁, the probability of an observer identifying an aliquot as containing one cell when the aliquot actually contains more than one cell;
μ, the mean number of cells in an aliquot; and
p, the probability a cell will grow into observable growth,
to evaluate the probability that the subsequent growth of an aliquot identified as having one cell is monoclonal.

14. The method of any of claims 1-13, wherein d) ii) comprises:
ii) fitting/applying the data values to a probability equation consisting of
$$P = \frac{2 {q}_{11}^{2} + 2 ( 1 - p ) {q}_{21}^{2} μ}{2 {q}_{11}^{2} + ( 2 - p ) {q}_{21}^{2} μ}$$
to evaluate the probability that the subsequent growth of an aliquot identified as having one cell is monoclonal.

15. The method of any of claims 1-14, wherein d) ii) comprises:
ii) fitting/applying the data values to a probability equation comprising unknowns consisting of the parameters listed in Table 7 to evaluate the probability that the subsequent growth of an aliquot identified as having one cell is monoclonal, wherein more than one (e.g. two, three, four, five, six, or more) sets of starting values for the unknowns are used to apply the data values to the probability equation.

16. The method of any of claims 1-15, wherein d) further comprises:
iii) assessing the evaluation of the probability using one or more statistical analyses, e.g. maximum likelihood, minimum sum of squares, minimum chi-squared, or log-likelihood ratio, wherein a higher maximum likelihood, lower minimum sum of squares, lower minimum chi-squared, and lower log-likelihood ratio indicate a more reliable evaluation of the probability.

17. The method of any of claims 1-16, wherein the identification of cells within aliquots of c) is accomplished using fluorescence microscopy.

18. A method of evaluating the reliability of a single cell cloning technique, comprising:
a) providing a solution comprising a population of cells;
b) performing a first estimate of the value of the probability of monoclonality of the single cell cloning technique, comprising:
i) forming a plurality of aliquots of the solution;
ii) identifying aliquots having one cell; and
iii) providing, for aliquots identified as having one cell, a value of the probability that subsequent growth was monoclonal,
c) practicing the single cell cloning technique for an interval,
d) performing a second estimate of the value of the probability of monoclonality of the single cell cloning technique, comprising:
i) forming a plurality of aliquots of the solution;
ii) identifying aliquots having one cell; and
iii) providing, for aliquots identified as having one cell, a value of the probability that subsequent growth was monoclonal; and
e) comparing the second estimate of the value of the probability of monoclonality of the single cell cloning technique to the first estimate or to a threshold value of the probability of monoclonality,
thereby evaluating the reliability of a single cell cloning technique.

19. The method of claim 18, wherein the method further comprises adjusting the single cell cloning technique to improve the value of the probability of monoclonality.

20. The method of either of claim 18 or 19, wherein b) ii) and d) ii) comprise identifying aliquots having zero, one, or more cells.

21. The method of any one of claims 18-20, wherein b) ii) and d) ii) comprise identifying aliquots having zero, one, or more cells using fluorescence microscopy.

22. The method of any one of claims 18-21, wherein b) ii) and d) ii) comprise a plurality of observers identifying aliquots having zero, one, or more cells using fluorescence microscopy.

23. The method of any one of claims 18-22, wherein b) ii) and d) ii) comprise two observers identifying aliquots having zero, one, or more cells using fluorescence microscopy.

24. The method of either of claim 22 or 23, wherein the observers identify an aliquot having zero, one, or more cells based on examining the same fluorescence micrograph of the aliquot.

25. The method of either of claim 22 or 23, wherein the observers identify an aliquot having zero, one, or more cells based on examining different fluorescence micrographs of the aliquot, e.g., a distinct fluorescence micrograph for each observer.

26. The method of any of claims 22-25, wherein the observers further identify whether an aliquot shows subsequent growth.

27. The method of any of claims 18-26, wherein b) iii) and d) iii) comprise:
a) calculating data values for the frequencies at which aliquots were identified as having zero, one, or more cells, and whether the aliquots showed or did not show subsequent growth; and
b) using a probability equation and the data values to evaluate the probability that the subsequent growth of an aliquot identified as having one cell is monoclonal.

28. The method of any of claims 18-27, wherein b) iii) a) and d) iii) a) comprise:
a) calculating data values for the frequencies at which aliquots were identified as having zero, one, or more cells, and whether the aliquots showed or did not show subsequent growth, the data values comprising the data values listed in Table 6.

29. The method of any of claims 18-28, wherein b) iii) a) and d) iii) a) comprise:
a) calculating data values for the frequencies at which aliquots were identified as having zero, one, or more cells, and whether the aliquots showed or did not show subsequent growth, the data values comprising:
n₀₁, the number of aliquots two observers identified as containing zero cells that did not show subsequent growth;
n₀₂, the number of aliquots one observer identified as containing zero cells and one observer identified as containing one cell that did not show subsequent growth;
n₀₃, the number of aliquots two observers identified as containing one cell that did not show subsequent growth;
n₀₄, the number of aliquots one observer identified as containing zero cells and one observer identified as containing more than one cell that did not show subsequent growth;
n₀₅, the number of aliquots one observer identified as containing one cell and one observer identified as containing more than one cell that did not show subsequent growth;
n₀₆, the number of aliquots two observers identified as containing more than one cell that did not show subsequent growth;
n₁₁, the number of aliquots two observers identified as containing zero cells that showed subsequent growth;
n₁₂, the number of aliquots one observer identified as containing zero cells and one observer identified as containing one cell that showed subsequent growth;
n₁₃, the number of aliquots two observers identified as containing one cell that showed subsequent growth;
n₁₄, the number of aliquots one observer identified as containing zero cells and one observer identified as containing more than one cell that showed subsequent growth;
n₁₅, the number of aliquots one observer identified as containing one cell and one observer identified as containing more than one cell that showed subsequent growth; and
n₁₆, the number of aliquots two observers identified as containing more than one cell that showed subsequent growth.

30. The method of any of claims 18-29, wherein b) iii) b) and d) iii) b) comprise:
b) fitting/applying the data values to a probability equation comprising unknowns consisting of the parameters listed in Table 6 to evaluate the probability that the subsequent growth of an aliquot identified as having one cell is monoclonal.

31. The method of any of claims 18-30, wherein b) iii) b) and d) iii) b) comprise:
b) fitting/applying the data values to a probability equation comprising unknowns consisting of:
q₀₀, the probability of an observer identifying an aliquot as containing zero cells when the aliquot actually contains zero cells;
q₁₀, the probability of an observer identifying an aliquot as containing zero cells when the aliquot actually contains one cell;
q₀₁, the probability of an observer identifying an aliquot as containing one cell when the aliquot actually contains zero cells;
q₁₁, the probability of an observer identifying an aliquot as containing one cell when the aliquot actually contains one cell;
q₂₁, the probability of an observer identifying an aliquot as containing one cell when the aliquot actually contains more than one cell;
μ, the mean number of cells in an aliquot; and
p, the probability a cell will grow into observable growth,
to evaluate the probability that the subsequent growth of an aliquot identified as having one cell is monoclonal.

32. The method of any of claims 18-31, wherein b) iii) b) and d) iii) b) comprise:
b) fitting/applying the data values to a probability equation consisting of
$$P = \frac{2 {q}_{11}^{2} + 2 ( 1 - p ) {q}_{21}^{2} μ}{2 {q}_{11}^{2} + ( 2 - p ) {q}_{21}^{2} μ}$$
to evaluate the probability that the subsequent growth of an aliquot identified as having one cell is monoclonal.

33. The method of any of claims 18-32, wherein b) iii) b) and d) iii) b) comprise:
b) fitting/applying the data values to a probability equation comprising unknowns consisting of the parameters listed in Table 7 to evaluate the probability that the subsequent growth of an aliquot identified as having one cell is monoclonal, wherein more than one (e.g. two, three, four, five, six, or more) sets of starting values for the unknowns are used to apply the data values to the probability equation.

34. The method of any of claims 18-33, wherein b) iii) and d) iii) further comprise:
c) assessing the evaluation of the probability using one or more statistical analyses, e.g. maximum likelihood, minimum sum of squares, minimum chi-squared, or log-likelihood ratio, wherein a higher maximum likelihood, lower minimum sum of squares, lower minimum chi-squared, and lower log-likelihood ratio indicate a more reliable evaluation of the probability.

35. The method of any of claims 18-34, wherein the single cell cloning technique is chosen from CACC, FACS, or spotting.

36. The method of any of claims 18-35, wherein the single cell cloning technique is CACC.

37. The method of any of claims 18-35, wherein the single cell cloning technique is FACS.

38. The method of any of claims 18-35, wherein the single cell cloning technique is spotting.

39. The method of any of claims 18-38, wherein the interval comprises a number of aliquots formed without evaluating a value of the probability of monoclonality.

40. The method of claim 39, wherein the number of aliquots is at least 1, 10, 50, 100, 200, 500, 1000, 1500, 2000, 2500, 3000, or more.

41. The method of any of claims 18-38, wherein the interval comprises a number of multi-well plates, e.g., 96-well plates, filled with aliquots without evaluating a value of the probability of monoclonality.

42. The method of claim 41, wherein the number of multi-well plates, e.g., 96 well plates, is at least 1, 5, 10, 15, 20, 25, 30, or more.

43. The method of any of claims 18-42, wherein the steps of the method take the form of:
a), b), [c), d), e)]ₙ
wherein [c), d), e)] is repeated n times, and wherein n is greater than or equal to 1.

44. The method of claim 42, wherein n is greater than or equal to 2, 3, 4, 5, 6, 7, 8, 9, or 10.

45. The method of any of claims 18-44, wherein e) comprises:
e) comparing the second estimate of the value of the probability of monoclonality of the single cell cloning technique to the first estimate.

46. The method of any of claims 18-44, wherein e) comprises:
e) comparing the second estimate of the value of the probability of monoclonality of the single cell cloning technique to a threshold value of the probability of monoclonality.

47. The method of any of claims 1-46, wherein an observer, or plurality of observers, is (are) human observer(s).

48. The method of any of claims 1-46, wherein an observer or plurality of observers, is (are) a machine observer(s).
